(19) United States
(12) Patent Application Publication
(10) Pub. No.: US 2024/0185213 A1
(43) Pub. Date: Jun. 6, 2024

(54) Storytelling app for children and elderly

(71) Applicant: Ghramae Johnson, London (GB)

(72) Inventor: Ghramae Johnson, London (GB)

(73) Assignee: My Magic Moments Ltd, London (GB)

(21) Appl. No.: 18/377,472

(22) Filed: Oct. 6, 2023

Related U.S. Application Data
(60) Provisional application No. 63/408,864, filed on Sep. 22, 2022.

(51) Int. Cl. G06Q 20/24; A61M 21/00; G06T 13/40; G09B 5/06; G10L 13/033

(52) U.S. Cl. CPC G06Q 20/24; A61M 21/00; G06T 13/40; G10L 13/033; G09B 5/065

(57) ABSTRACT

The invention introduces a transformative storytelling platform tailored for both children and the elderly, combining advanced AI techniques for enhanced personalization. Users can transform photos into ultra-realistic 3D avatars that animate in sync with narratives, bolstered by voice cloning capabilities, obviating repetitive recording. The platform stands out with its capacity to involve relatives in storytelling, deepening familial ties and diversifying content. An internal library provides a plethora of stories, accommodating diverse user preferences. Moreover, the app integrates a unique credit system, granting users the discretion to dictate avatar screen-time, optimizing credit use. Beyond mere entertainment, this platform doubles as a therapeutic and developmental tool, incorporating elements beneficial for cognitive engagement. Altogether, this invention signifies a leap in digital storytelling, amalgamating cutting-edge technologies with deeply human-centric features, bridging generational divides and catering to emotional, cognitive, and social needs

### CROSS-REFERENCE TO RELATED APPLICATIONS

The present invention claims the priority of the U.S. provisional patent application No. 63/408,864 filed on Sep. 22, 2022. A petition to restore the priority of the provisional patent application No. 63/408,864, will be filed.

### STATEMENT REGARDING FEDERALLY SPONSORED RESEARCH OR DEVELOPMENT

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

### Field of the Invention

The present invention pertains to the domain of mobile and web applications, more specifically to an advanced storytelling platform tailored for both children and the elderly. This app harnesses state-of-the-art technologies, including Artificial Intelligence (AI), to transmute photos into ultra-realistic 3D animated avatars and employ voice cloning for a uniquely personal and interactive storytelling milieu. It delves into digital content delivery, enabling users to handpick tales from a broad in-app library and personalize their on-screen storytellers using their photos and voice inputs. A standout feature is the system facilitating the invitation of relatives, enriching storytelling content while bridging familial bonds. Furthermore, the invention traverses the realm of e-commerce, unveiling a nuanced credit mechanism, giving users the reigns to decide avatar display duration and judiciously manage in-app financial transactions, thus augmenting user involvement and revenue avenues

### Description of the Related Art

Current Landscape & Limitations: Digital storytelling apps have witnessed rapid evolution, fueled by the exponential growth in mobile technology and digital content consumption. These apps have primarily catered to the younger demographic, leveraging animated graphics and sound effects to engage children. The elderly, a demographic equally, if not more, inclined towards storytelling, have largely been overlooked. Existing apps often employ two-dimensional (2D) avatars that, though entertaining, are rudimentary and lack depth, rendering them less engaging for prolonged use.

Additionally, these 2D avatars follow predefined movements and expressions, which often cannot be customized. This restricts users from having a fully personalized experience and can sometimes lead to disengagement, especially when the target audience is looking for more immersive experiences.

The storytelling apps currently available on the market offer a limited selection of avatars, most of which are generic and cannot be tailored to user preferences. The personal touch, often a crucial element in storytelling, is thus lost. Moreover, while advancements like the ‘Talking Photo’ technology have been groundbreaking, they usually function in silos and aren't incorporated into the broader context of storytelling platforms.

Voice technology has seen immense progress, especially in the domains of voice recognition and cloning. However, such state-of-the-art technologies remain conspicuously absent from storytelling platforms. The possibility of having a loved one's voice or even one's voice cloned and used for storytelling purposes remains largely unexplored, depriving users of a deeply personal and emotionally resonant experience.

User Experience & Economic Aspects: Most storytelling apps follow a fixed pricing model where users pay a predetermined amount to access content. Such a model, while straightforward, doesn't offer flexibility. Users might feel compelled to engage with the app to “get their money's worth,” rather than genuinely enjoying the experience. A system that allows users to pay based on usage or desired features can offer more value and personalization.

Furthermore, the social aspect of storytelling, an integral part of the human experience, is notably absent from existing platforms. The idea of involving relatives, friends, or acquaintances in the storytelling process, whether by using their voices, avatars, or even including their stories, is not widely adopted, thus missing out on a layer of personal connection and shared experience.

Need for Holistic Development & Therapeutic Engagement: For children, storytelling is not merely a form of entertainment. It plays a vital role in their cognitive, emotional, and psychological development. Existing apps rarely address these multifaceted developmental needs in their content and delivery. On the other hand, for the elderly, storytelling can serve as a therapeutic tool, aiding memory recall, emotional well-being, and cognitive functioning. A platform that integrates simple arithmetic calculations or narrative elements can serve dual purposes—entertainment and therapeutic engagement.

Given these gaps in the current digital storytelling landscape, there is a clear and present need for a platform that not only offers advanced technological features but also addresses the emotional, social, and developmental aspects of storytelling. The proposed invention, with its myriad innovative features and user-centric approach, seeks to bridge these gaps and set a new standard in digital storytelling

### SUMMARY OF THE INVENTION

The invention pertains to a comprehensive storytelling platform available as mobile and web applications, specifically crafted for children and the elderly. Seamlessly integrating advanced technologies, this platform offers an unparalleled, personalized storytelling experience with a vision to stimulate developmental growth and provide therapeutic advantages.

Ultra-Realistic 3D Avatars, 2D Talking Photos & Face Swap: Leveraging state-of-the-art AI, the application transforms user-uploaded photographs into lifelike 3D avatars and 2D animated photos that can lip-sync to the narrative, reminiscent of “Talking Photo” technology. Additionally, the platform boasts transformative face swap capabilities, adding a layer of personalization.

Voice Cloning & Selection: The platform empowers users with choices: they can opt for a narrator from the rich internal library or customize their own using a photo and a pre-selected voice. With its voice cloning innovation, users can either record or replicate their voice for ensuing sessions, negating repetitive readings.

Inviting Relatives & Personalized Storytelling: A standout feature is the system that allows relatives to enhance the storytelling journey. By accepting an invitation, they gain access to the expansive story catalog and the option to acquire credits, leading to their photo's animation into an on-screen narrator. Such bespoke stories are instantly accessible, fostering familial ties.

Credit System & Avatar Duration Control: The platform introduces a strategic credit mechanism where costs correspond with the avatar's on-screen time, granting users the flexibility to dictate the avatar's duration, ensuring efficient credit management.

Diverse Story Library & User Engagement: An abundant internal library ensures the platform meets diverse storytelling appetites, enhancing engagement and delivering a dynamic experience for all age groups.

Therapeutic & Developmental Benefits: Transcending mere entertainment, this invention doubles as a therapeutic tool, especially beneficial for the elderly, embedding elements like basic arithmetic. For children, it champions holistic growth, addressing a spectrum of developmental facets.

Accessibility & User Experience: Designed for effortless navigation, the platform ensures accessibility and convenience for users across age brackets.

In sum, this invention synergizes cutting-edge AI technologies with user-centric design, aiming to not only entertain but also bridge generational divides, all while addressing the developmental and therapeutic requisites of its diverse audience.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention.

This invention brings forth a revolutionary storytelling platform encapsulated within web and mobile applications, adeptly catering to the diverse age groups of children and the elderly. Integrating a spectrum of advanced technologies, the platform offers an enriched and personalized storytelling experience, with an emphasis on fostering developmental growth and providing therapeutic engagement.

The application uses the following technologies to produce animated avatars that narrate and read the children's stories and games, and stories and articles for the elderly:
Ultra-Realistic 3D Lip-Syncing Animated Avatars.
2D Animated Avatars from Photos (Talking Photo Technology).
Face Swap Technology, used to swap the face of a 3D avatar with a face from a photo.
Voice cloning technology.

These technologies can be used together or separately, to create talking avatars that narrate to speak alongside and interact with a background video or still illustration presentation.

### 1—Ultra-Realistic 3D Lip-Syncing Animated Avatars

The crux of this innovation is the generation of hyper-realistic 3D avatars. Through advanced AI algorithms, the application creates lifelike 3D avatars that can enact anatomical movements such as blinking, head movements, facial expressions, and more. Their most striking feature is their ability to lip-sync accurately in accordance with both text-to-speech solutions and pre-recorded or streamed audio.

This offers immense flexibility in terms of customization. Users have the choice to modify various assets of these avatars, including but not limited to, hair, clothing, eyeglasses, tattoos, hats, and piercings. The animated avatars can represent a diverse range of entities including humans, animals, fruits, characters, toys, and other imaginative creatures.

The versatility of these 3D avatars extends to their positioning within the storytelling environment. They can seamlessly integrate into the story video, be it alongside, overlaid, or even prerecorded within the video. Their presentation isn't limited either—they can appear without a background, within specific geometric confines like circles or boxes, or even with custom backgrounds.

2. 2D Animated Avatars from Photos (Talking Photo Technology)

Marrying the appeal of photographs with the vitality of animations, the application employs the “Talking Photo” technology. This technique animates photos of entities ranging from humans and animals to objects and characters, making them appear as if they are speaking. This talking illusion is achieved by syncing the animated photos with a text-to-speech solution or even with pre-recorded or live-streamed audio. The animated representations can vary, displaying just the head, a combination of the head and torso, or the full body. These 2D avatars can also be strategically positioned based on creative preference, ensuring they complement the background video or still illustration effectively.

### 3. Face Swap Technology

The Face Swap functionality in the application is nothing short of transformative. Using advanced deep fake techniques, the software allows users to superimpose a chosen face onto the 3D avatars, the talking photos, or even characters within the story video. This means that any avatar or character can be personalized to resemble a desired individual, allowing for a higher degree of customization and personal connection. This feature ensures that users can truly see themselves or their chosen entities in the story, enhancing immersion and engagement.

### 4. Voice Cloning Technology:

Narration forms the backbone of any storytelling session. Recognizing this, the application integrates state-of-the-art AI voice cloning and text-to-speech technologies. Users can record their voices, which the application then clones. This cloned voice can be used to narrate stories, articles, books, and more, bringing a familiar auditory element to the narration. Whether it's for children who find comfort in their parent's voice or the elderly who prefer a familiar voice narrating their daily newspaper, this technology bridges the gap between digital storytelling and personal touch.

### 3. Inviting Relatives & Personalized Story Creation:

The platform introduces a unique system enabling users to invite relatives to participate in the storytelling experience. Upon acceptance, the invited relatives gain access to a diverse story library and the option to purchase credits. These credits facilitate the conversion of their photographs into on-screen storytellers, thereby creating personalized stories that are instantly accessible to the child's account, fostering a sense of connection and familial bonding.

### 4. Credit System & Duration Control:

An integral component of the invention is the introduction of a credit system. Users are charged based on the chosen duration of the avatar's on-screen appearance, with options for 30 seconds, 1 minute, or the full length of the story. This feature allows users to exercise control over credit usage, thereby managing the overall expenditure efficiently.

### 5. Diverse Internal Library & User Engagement:

The application boasts a vast and varied internal library of stories, ensuring the availability of content that caters to a wide range of user preferences and interests. This extensive collection is instrumental in fostering sustained user engagement and providing a dynamic and enriching storytelling experience for both children and the elderly.

### 6. Therapeutic & Developmental Engagement:

The platform extends beyond mere entertainment, serving as a valuable tool for learning therapy, especially for the elderly. By incorporating elements such as simple arithmetic calculations and diverse narratives, it aids in cognitive stimulation. For children, the platform contributes to holistic development, addressing various aspects including psychological, mental, emotional, educational, and social growth.

### 7. Accessibility & User-Friendly Interface:

The platform ensures ease of access and navigation through a user-friendly interface. Whether it's selecting stories, customizing avatars, or inviting relatives, each feature is designed with the user's convenience in mind, making the platform accessible to individuals across different age groups.

In summary, this invention amalgamates cutting-edge AI technologies with user-centric features, creating a versatile and immersive storytelling platform. Its capabilities extend to bridging generational gaps, fostering familial connections, and supporting the developmental and therapeutic needs of its diverse user base, thereby redefining the storytelling experience for children and the elderly.

### 8. User Flow and Application

### For Children:

Upon launching the application, either the parent or child is greeted with a user-friendly interface. They can log in to their account, subsequently entering an expansive library of Virtual Readers. Here, they're offered an array of choices between captivating 3D avatars or the innovative Talking Photos. Once the preferred avatar is selected, they navigate to the story section. This is where they are met with a plethora of animated or illustrated stories and interactive games. But the zenith of this digital journey is attained when they hit ‘play’. In an instant, the story or game bursts to life, flawlessly synchronized with the chosen avatar, culminating in an unparalleled storytelling adventure.

### For the Elderly:

Tailored meticulously for an older demographic, the application provides an intuitive experience. Post login, elderly users can peruse through the Virtual Reader selection, subsequently opting for their desired reading material. This could range from heartwarming stories and daily newspapers to enlightening books and riveting articles. And as they press ‘play’, the magic unfolds. Their selected material harmoniously aligns with the avatar or narrator, crafting a seamless, immersive reading journey.

The proposed invention stands as a beacon of innovation in the realm of digital storytelling. With its ultra-realistic 3D and 2D avatars, transformative face swap technology, and personalized voice cloning capabilities, it redefines the way stories are told and experienced. Whether it's children immersing themselves in enchanting tales or the elderly reconnecting with the joy of narration, this application promises an unmatched storytelling voyage

1. A storytelling application platform enabling the conversion of user-uploaded photographs into animated, lip-syncing avatars for personalized storytelling experiences to both children and the elderly.

2. The platform of claim 1, where the animated avatars are created using advanced AI technology, analogous to talking photo technology.

3. A method within the platform of claim 1 for voice cloning, allowing users to clone their own voice for personalized storytelling, with the option to store and reuse the cloned voice for future sessions.

4. The method of claim 3, wherein users can select a pre-recorded voice from a library or record their own voice within the application.

5. A system within the platform of claim 1, enabling users to invite relatives to participate, granting invited users access to the story library and the ability to purchase credits for creating and sharing personalized stories.

6. The system of claim 5, wherein invited relatives can convert their photographs into on-screen storytellers, enhancing the personalized experience and fostering familial bonding.

7. A credit system integrated within the platform of claim 1, allowing users to control the duration of the avatar's on-screen appearance, with selectable durations and associated costs.

8. The credit system of claim 7, wherein users can select the avatar to appear on the screen for durations of 30 seconds, 1 minute, or the full length of the story, with associated costs per minute.

9. The platform of claim 1, further comprising an internal library of stories, facilitating a diverse range of user preferences and promoting sustained engagement.

10. A method for utilizing the platform of claim 1 for cognitive stimulation and developmental engagement, supporting the psychological, mental, emotional, educational, and social growth of children and the elderly.

11. A user-friendly interface in the platform of claim 1, ensuring accessibility and convenience for users across different demographics.

12. A method for utilizing the platform of claim 1 to foster intergenerational connections, by allowing both children and the elderly to experience personalized storytelling from familiar figures.

13. A system within the platform of claim 1, for managing and purchasing credits to control the appearance duration and features of the animated avatars, thereby allowing users to manage their expenditure on the platform

14. A face swap technology within the platform of claim 1 using advanced deep fake techniques, the technology allows users to superimpose a chosen face onto the 3D avatars, the talking photos, or even characters within the story video, wherein any avatar or character can be personalized to resemble a desired individual, allowing for a higher degree of customization and personal connection.
